(19) United States
(12) Patent Application Publication
(10) Pub. No.: US 2025/0314562 A1
(43) Pub. Date: Oct. 9, 2025

(54) ALCOHOL TESTING DEVICE

(71) Applicant: Brave NMI Williams, Bangor, ME (US)

(72) Inventor: Brave NMI Williams, Bangor, ME (US)

(21) Appl. No.: 18/628,681

(22) Filed: Apr. 6, 2024

(51) Int. Cl. G01N 1/14; G01N 1/34

(52) U.S. Cl. CPC G01N 1/14; G01N 1/34

(57) ABSTRACT

An alcohol testing device is designed for the filtration, retention, and controlled release of liquid samples for specific gravity measurement. The chamber comprises a transparent body with top and bottom ends, accommodating a device for checking alcohol content. At the bottom end, a filter facilitates liquid sample filtration, while a narrowed opening enables liquid entrapment under vacuum. The top end features an opening for air expulsion and may be sealed by a finger, thumb, or a user-actuated device like a stopper to create a vacuum, thereby trapping the liquid sample. This design enhances the efficiency of obtaining specific gravity measurements, particularly with samples containing large suspended solid bodies.

### CROSS-REFERENCE TO RELATED APPLICATIONS

Not Applicable

### STATEMENT FOR FEDERALLY FUNDED RESEARCH AND DEVELOPMENT

Not Applicable

### BACKGROUND OF THE INVENTION

Brewing wine, beer, and kombucha has garnered significant interest due to its cost-effectiveness, flavor control, and creative appeal. However, a crucial aspect of many brewing processes involves monitoring the alcohol content of the beverage, often requiring multiple checks per day. Traditionally, this process involves collecting a liquid sample, filtering it, transferring it to a graduated cylinder, and then using a specialized device to measure alcohol content based on flotation.

Particularly in brewing processes involving ingredients like red grape skins or beer, there are often large particles or solids that must be filtered out before the liquid sample can be tested. These solid bodies can pose challenges, such as clogging the inrush hole of the testing devices and impeding the fluid flow. This testing process can be laborious and time-consuming, requiring the use of various equipment such as a ladle, filter screen, funnel, and graduated cylinder, especially when dealing with samples containing significant solid particles.

While some testing devices have been developed for swiftly collecting samples from clear liquids, they often encounter issues with clogged valves when handling non-clear liquids and lack filtration capabilities during testing. Furthermore, these devices typically cannot be disassembled for cleaning, adding to their limitations.

Traditional methods such as the “wine thief” utilize vacuum extraction to obtain small samples for testing, but they lack built-in filtration mechanisms and do not incorporate a device for checking alcohol content for testing within the device itself.

Presently, there exists no single system within the brewing domain that seamlessly combines rapid filtration, efficient sample collection, and accurate alcohol testing. This highlights the need for an alcohol testing device that quickly filters and extracts liquid samples, securely retains the liquid during alcohol content testing, and facilitates prompt release. Moreover, such a testing device should be designed with ease of disassembly and cleaning in mind to address the challenges posed by cleaning processes.

### BRIEF SUMMARY OF THE INVENTION

The invention is an alcohol testing device designed for efficient filtration, retention, and controlled release of liquid samples for alcohol content measurement. The chamber is transparent and features a top and bottom end. At the bottom end, a filter facilitates liquid sample filtration, while an opening at the top end expels air from the chamber and can be sealed to create a vacuum, effectively trapping the liquid sample within. Additionally, a narrowed opening at the bottom end, connected to the filter, enables liquid entrapment within the chamber under vacuum conditions, facilitating both liquid inflow and outflow. The top end is equipped with a narrowed opening for air release, allowing for control by finger or thumb to regulate the inflow, retention, and expulsion of fluids.

Another example of an alcohol testing device features a rapid-disengage stopper mechanism, capable of releasing air from the chamber upon fluid influx and sealing the top opening to create a vacuum, facilitating sample analysis. This integrated design enhances testing efficiency by combining filtration, sample collection, fluid management, and alcohol testing functionalities within a single apparatus. Additionally, components such as the top and/or bottom caps, and filter, are designed for easy disassembly and reassembly to facilitate cleaning procedures, offering advantages not readily discernible by those skilled in the art.

### BRIEF DESCRIPTION OF THE DRAWINGS

FIG. 1 presents a frontal view of an alcohol testing device in accordance with one embodiment of the present disclosure.

FIGS. 2 and 3 depict frontal views schematically illustrating the alcohol testing device showcased in FIG. 1, with FIG. 2 demonstrating regulation by a thumb to control the inflow, retention, and expulsion of fluids.

FIGS. 4 and 4A offer an exploded view and a detailed sectional view, respectively, showcasing an alternative example of an alcohol testing device as described in various embodiments of the present invention.

FIGS. 5 and 5A provide an exploded view and a detailed sectional view, respectively, presenting another alternate example of an alcohol testing device described in various embodiments of the present invention.

FIGS. 6 and 6A display an exploded view and a detailed sectional view, respectively, illustrating yet another alternate example of an alcohol testing device as described in various embodiments of the present invention.

### DETAILED DESCRIPTION OF THE EMBODIMENTS

The following detailed description pertains to several embodiments of the technology disclosed herein. These embodiments are provided to enable a thorough understanding of the technology and its various aspects. It should be noted that while specific embodiments are described, variations and modifications may be made without departing from the scope of the appended claims. The identified components within the described embodiments are terms of art that may vary and should not limit the present disclosure. In the following sections, a detailed description of several embodiments of the technology is provided. Each embodiment is described in sufficient detail to enable one skilled in the art to practice the concepts disclosed.

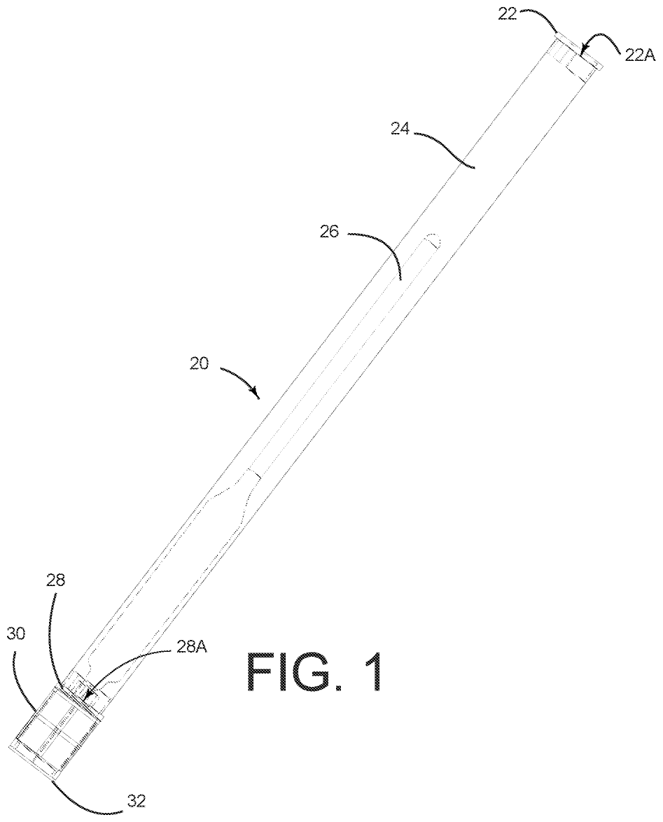

In the accompanying drawings, identical reference numerals denote similar elements across different views, FIG. 1 depicts the preferred embodiment of the alcohol testing device 20. The device comprises a top-end cap 22, featuring a hole 22A to facilitate the ingress of a liquid into a chamber 24. At the opposite end, a bottom-end cap 28 is equipped with a hole 28A of sufficient size to allow liquid entry while maintaining the capability for vacuum creation when the hole 22A in the top-end cap 22 is sealed by a finger or thumb. The transparent chamber 24, typically constructed from FDA-compliant and BPA-free nonreactive acrylic material, serves as the vessel for holding the liquid sample during testing procedures. Within this chamber, a device for checking alcohol content 26 can be suspended in the liquid. Integral to the bottom-end cap 28 is a filter 30, which serves to intercept and prevent the entry of large particles into the chamber 24, thus ensuring the integrity of the sample being tested and preventing clogging of the bottom-end hole 28A in the bottom-end cap 28. Each component, including the top-end cap 22, bottom-end cap 28, filter 30, and filter cap 32, is designed for ease of removal, facilitating simplified cleaning and disinfection processes.

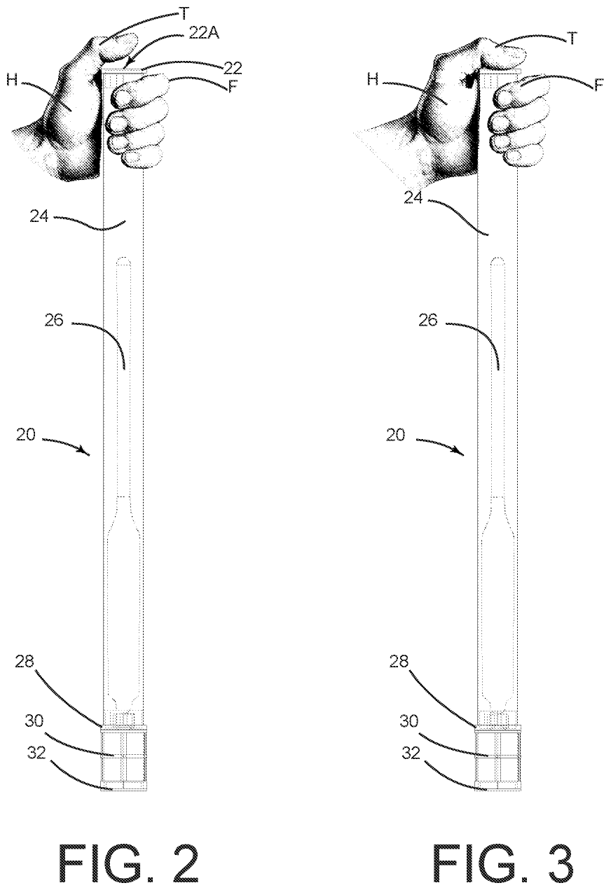

With specific regard to FIGS. 2 and 3, the operation of this embodiment of the alcohol testing device 20 involves manual manipulation by a human hand (H). The user grasps the upper end of the chamber 24 while submerging the lower end into the liquid until the device for checking alcohol content 26 achieves buoyancy. Subsequently, the user can employ either their thumb (T) or finger (F) to cover the hole 22A located in the upper cap 22. This action initiates the creation of a vacuum within the chamber 24, effectively retaining the liquid for a duration sufficient to obtain a reading from the device for checking alcohol content 26.

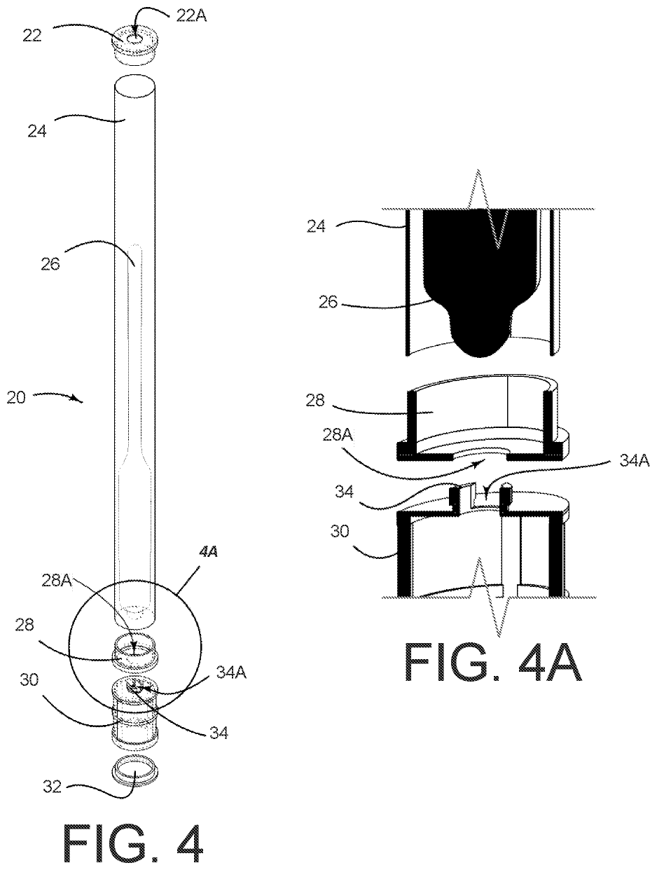

Referring now to FIGS. 4 and 4A, which present an exploded view of the testing device 20, the upper-end cap 22 is equipped with a hole 22A designed to facilitate the escape of air and subsequently be sealed once the chamber 24 is filled with liquid. The upper-end cap 22 may be inserted into the chamber 24 via friction, as can be the case with the bottom-end cap 28 fitting into the bottom portion of the chamber 24. The bottom-end cap 28 allows for the ingress of liquid through the hole 28A. In this particular embodiment, the filter 30 is distinct from the bottom-end cap, although alternative configurations may integrate the filter 30 and the bottom-end cap into a single unit. The filter cap 32 may be removable, although other embodiments may feature a filter that is a single, inseparable component. In this embodiment, the top of the filter 34 includes a retaining ring that is split to form a gap 34A, serving to prevent the bottom end of the device for checking alcohol content 26 from obstructing the hole 28A in the bottom-end cap 28.—

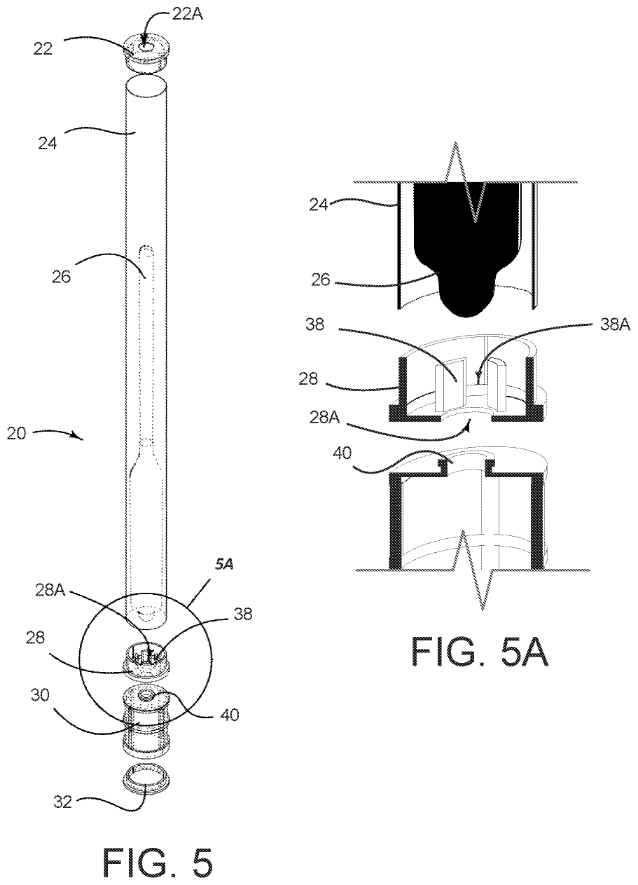

As depicted in FIGS. 5 and 5A, the testing device 20 may feature a distinct configuration of the bottom cap 28 to facilitate the inflow of fluid into the chamber 24. In this particular embodiment, the bottom-end cap 28 incorporates a plurality of protuberances 38 extending upward, serving the purpose of enabling the ingress of liquid into through the hole 28A in the bottom-end cap 28 and ultimately into the chamber 24. Additionally, there may exist a retaining ring 40 positioned at the top of the filter 30, facilitating the attachment and detachment of the filter 30 from the bottom-end cap 28.

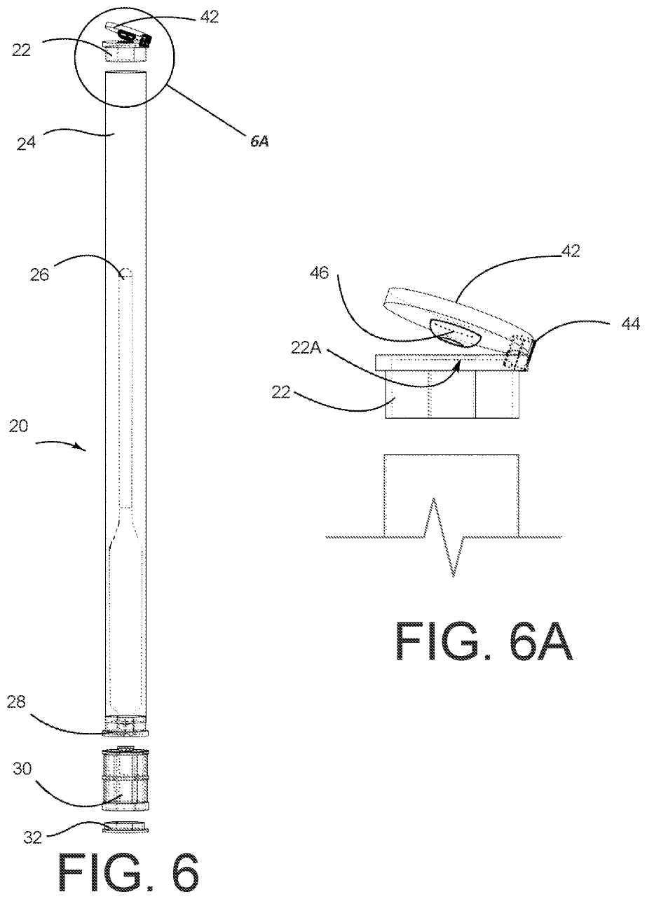

As illustrated in FIGS. 6 and 6A, an alternative embodiment of the upper-end cap 22 may incorporate a second cap 42 affixed to the upper-end cap 22 via a hinging member 44. In this configuration, the hole 22A in the upper-end cap 22 may be sealed using a protrusion 46 that serves to plug the hole 22A in the upper-end cap 22, rather than relying on manual obstruction by a finger or thumb. Various other configurations may be employed in alternative embodiments, provided that the upper end is designed to facilitate the escape of air while enabling restriction to create a vacuum within the chamber 24.

The above description is presented for illustrative purposes, and it will be apparent to those skilled in the art that various modifications and variations may be made without departing from the scope of the invention. All such modifications are intended to be included within the scope of the following claims.

What is claimed is:

1. An alcohol testing device comprising:
A transparent chamber designed to accommodate an alcohol testing device, featuring a top and bottom end;
a filter situated at the bottom end to facilitate liquid sample filtration;
a narrowed opening at the bottom end, communicating with the filter, facilitating liquid entrapment within the chamber under vacuum, and enabling liquid inflow and outflow; and,
an opening at the top end capable of expelling air from the chamber and is sealable to create a vacuum, thereby trapping the liquid sample within the chamber.

2. The alcohol testing device of claim 1, wherein the top end is equipped with a narrowed opening for air release, allowing control by finger or thumb to regulate the inflow, retention, and expelling of fluids.

3. The alcohol testing device of claim 1, featuring a rapid-disengage stopper mechanism capable of releasing air from the chamber upon fluid influx and sealing the top opening to create a vacuum for liquid sample entrapment, facilitating sample analysis.

4. The alcohol testing device of claim 2, wherein the integrated design enhances testing efficiency by combining filtration, sample collection, fluid management, and alcohol testing functionalities within a single apparatus.

5. The alcohol testing device of claim 3, wherein the integrated design enhances testing efficiency by combining filtration, sample collection, fluid management, and alcohol testing functionalities within a single apparatus.

6. The alcohol testing device of claim 4, wherein components such as the top and/or bottom caps, and filter, are configured for easy disassembly and reassembly to facilitate cleaning procedures.

7. The alcohol testing device of claim 5, wherein components such as the top and/or bottom caps, and filter, are configured for easy disassembly and reassembly to facilitate cleaning procedures.
